(19) United States
(12) Plant Patent Application Publication
(10) Pub. No.: US 2009/0271901 P1
(43) Pub. Date: Oct. 29, 2009

(54) AGASTACHE PLANT NAMED 'SUMMER LOVE'

(50) ;

(75) Inventor: Janet N. Egger, Wilsonville, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 12/150,186

(22) Filed: Apr. 25, 2008

(51) Int. Cl. A01H 5/00

(52) U.S. Cl. PLT/399

(57) ABSTRACT

A new and distinct form of Agastache plant characterized by large, vivid purple flowers in large clusters, long bloom time, upright plant habit with dense branching, and excellent vigor.

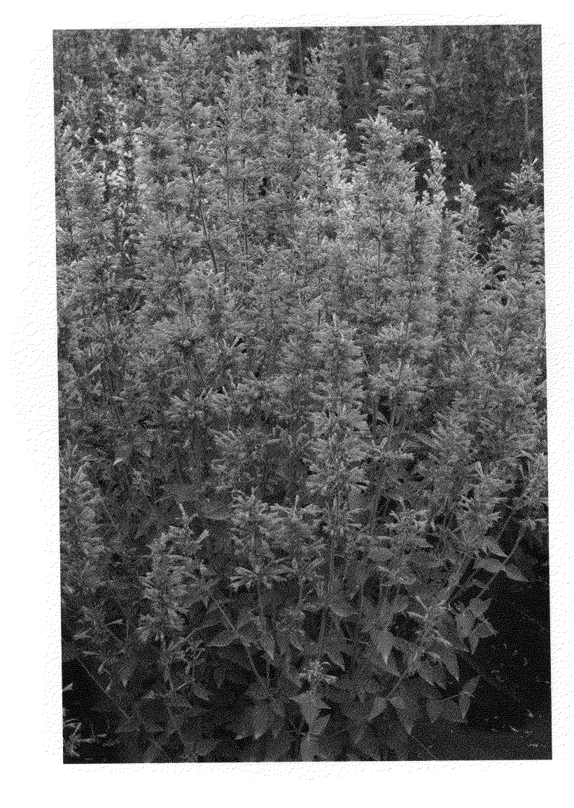

### BOTANICAL DENOMINATION

Agastache spp.

### VARIETY DESIGNATION

‘Summer Love’

### BACKGROUND OF THE INVENTION

The present invention relates to a new and distinct cultivar of Agastache, given the name, ‘Summer Love’. Agastache is in the family Lamiaceae. The new cultivar is a F2 selection from a planned breeding program. The original cross was between a dwarf form of Agastache pallidiflora ‘Pink Pop’ (an unpatented plant) as the seed parent and Agastache cana dark pink (an unpatented plant) as the pollen parent.

This plant exhibits the following characteristics:
1. Large, vivid purple flowers in large clusters,
2. Upright plant habit with dense branching,
3. Long bloom time,
4. Excellent vigor.

The new variety has been reproduced only by asexual propagation (cuttings and micropropagation). Each of the progeny exhibits identical characteristics to the original plant. Asexual propagation by division and micropropagation as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The drawing shows a one-year-old Agastache ‘Summer Love’ growing in the garden in late summer in Canby, Oreg.

### DETAILED PLANT DESCRIPTION

The following is a detailed description of the new Agastache cultivar based on observations of two-year-old specimen grown outside in the ground in the garden in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart.
Plant:
   Form.—upright, herbaceous perennial.
   Cold hardiness.—USDA Zone 6-9.
   Size.—grows to 90 cm tall and 60 cm wide.
   Vigor.—excellent.
   Stem.—square, grows to 8 mm wide and 60 cm tall, internodes average 6 cm long, Yellow Green 147B.
   Roots.—White 155B, fibrous, plants root easily from stem tip cuttings.
Leaf:
   Type.—simple.
   Arrangement.—opposite.
   Shape.—ovate.
   Venation.—pinnate.
   Margins.—crenate.
   Apex.—obtuse.
   Base.—cordate.
   Blade size.—grows to 4.2 cm long and 2.4 cm wide.
   Surface texture.—puberulous on both sides.
   Petiole description.—grows from 2.6 cm long to 0.9 cm long and 1.4 mm wide, puberulous, Yellow Green 147C.
   Leaf color.—Top side — closest to Yellow Green 147A, but darker Bottom side — Yellow Green 147B.
Inflorescence:
   Type.—terminal spike.
   Number of flowers.—about 190 per spike in 9 to 25 flowered, dense whorls.
   Peduncle.—20 cm to 30 cm tall and 2 mm wide, pubescent, Yellow Green 147B.
   Pedicel.—1 mm to 4 mm long, pubescent, color closest to Greyed Orange 177A, but more purple.
   Bloom period.—June through frost in Canby, Oreg.
Flower bud:
   Size.—17 mm long and 3 mm wide at the widest point prior to opening.
   Description.—tubular.
   Surface texture.—pubescent.
   Color.—Greyed Purple 187C.
Flower:
   Type.—zygomorphic.
   Shape.—bilabiate, tubular.
   Size.—3 cm long and 1 cm wide.
   Corolla description.—3 cm long and spreads to 1 cm wide, color Red Purple 72A inside and out, pubescent inside and out, tube 2.3 cm long and 1 mm to 4 mm wide, 2 upper lobes joined 3 mm long and 4 mm wide, acute, entire, three lower lobes, two spreading sideways, the terminal lobe broad, reflexed, obcordate and mucronate, entire, 6 mm wide and 5 mm deep, two side lobes 3 mm long and 2.5 mm wide.
   Calyx description.—7 mm long and 3 mm wide, color between Greyed Purple 187A and Gray 201A, pubescent outside and glabrous inside, 5 lobed, lobes narrow 3 mm long, entire, acuminate.
   Pistil description.—36 mm long, ovary 0.5 mm long, Yellow Green 146C, style and stigma 35.5 mm long, Red Purple 72A at the tip to White 155D at base.
   Stamen description.—4 in number, paired, exerted and reflexed, filaments 7 mm long, Red Purple 72A, anthers 1 mm long, Violet 83A, pollen White 155D.
   Fragrance.—strong, herbal.
   Lastingness.—a spike blooms for about 3 weeks on the plant.
Fruit: 4 nutlets, Green 144A
Seed: oblong, 1.5 mm long, Black 202A, fertile
Pests and diseases: Agastache are known to be susceptible to mildew and rust in dry summer months. The new cultivar has no known resistances but has shown no problems in Canby, Oreg.
Comparisons: Compared to Agastache pallidiflora ‘Pink Pop’ dwarf, the seed parent, the new variety has brighter purple flowers with darker calyces, larger flowers and inflorescences. Compared to Agastache cana dark pink, the pollen parent, the new variety has denser inflorescences, with brighter, more purple, flower color, and a more compact habit. Compared to Agastache ‘Kiegapur’ (U.S. Plant Pat. No. 16,067), the new variety has darker purple flowers and a taller, denser habit.

I claim:

1. A new and distinct form of Agastache plant substantially as shown and described.
